(19) United States
(12) Patent Application Publication
(10) Pub. No.: US 2024/0165173 A1
(43) Pub. Date: May 23, 2024

(54) METHOD OF REDUCING URIC ACID BY USING LACTOBACILLUS PARACASEI LT12

(71) Applicant: SHANGHAI LYTONE BIOCHEMICALS, LTD., SHANGHAI (CN)

(72) Inventors: XIANGRU KONG, SHANGHAI (CN); WEITING TSENG, NEW TAIPEI CITY (TW); WEITING CHANG, NEW TAIPEI CITY (TW); WILLIAM T. H. CHANG, NEW TAIPEI CITY (TW)

(21) Appl. No.: 18/512,470

(22) Filed: Nov. 17, 2023

(30) Foreign Application Priority Data
Nov. 17, 2022 (CN) 202211458978.3

(51) Int. Cl. A61K 35/747; A61P 13/12

(52) U.S. Cl. CPC A61K 35/747; A61P 13/12; C12R 2001/25

(57) ABSTRACT

The present disclosure provides a method for reducing uric acid by using Lactobacillus paracasei LT12 or Lactobacillus plantarum CBT LP3. The technical schemes provided herein produces the efficacies in reducing uric acid and protecting kidneys, wherein Lactobacillus paracasei LT12 and Lactobacillus plantarum CBT LP3 can effectively degrade inosine and guanosine and reduce production of uric acid, serum creatinine, and blood urea nitrogen; reducing renal pro-inflammatory factor levels; decreasing oxidative damage caused by uric acid production; and promoting uric acid excretion. Mice experiments prove that mice with high uric acid levels, after continuous consumption of the composition of the present invention, have significantly lowered blood uric acid levels. In addition, compared to control group, renal damage is significantly reduced, indicating an improved efficacy in renal protection.

### FIELD OF THE INVENTION

The invention relates to the method of reducing uric acid by using Lactobacillus paracasei LT12.

### BACKGROUND OF THE INVENTION

Hyperuricemia (HUA) is a general metabolic problem caused by dysfunction of purine metabolism, characterized by elevated blood uric acid levels. Under normal purine diet, fasting blood uric acid levels are detected twice on different days. If the level is higher than 420 μmol/L for men or 360 μmol/L for women, the person is determined to suffer from hyperuricemia. Hyperuricemia usually accompanies with chronic, low degree of inflammation. For long-term hyperuricemia, various complications such as gout, diabetes, metabolic syndrome, oxidative stress, inflammation, hypertension, and endothelial function disorder may be induced. In addition, severe burden will be caused to the kidneys, leading to kidney injuries.

In recent years, high purine diet increases as the levels of material life of people increase. The number of hyperuricemia patients gradually increases each year. There is a great need for the development of relevant products that address the hyperuricemia problem.

### SUMMARY OF THE INVENTION

To solve the above-mentioned technical problem, that present invention provides probiotics that are efficacious in reducing uric acid levels, protecting kidney and immune modulation, and compositions comprising the same.

For the above purposes, the present invention provides technical schemes as listed below.

Technical scheme 1: A method of reducing uric acid levels by using Lactobacillus paracasei LT12.

Technical scheme 2: A method of reducing uric acid levels by using Lactobacillus plantarum CBT LP3.

Technical scheme 3: A method of reducing uric acid levels by using a combination of Lactobacillus paracasei LT12 and Lactobacillus plantarum CBT LP3.

Technical scheme 4: A method of protecting kidney by using Lactobacillus paracasei LT12.

Technical scheme 5: A method of protecting kidney by using Lactobacillus plantarum CBT LP3.

Technical scheme 6: A method of protecting kidney by using a combination of Lactobacillus paracasei LT12 and Lactobacillus plantarum CBT LP3.

Technical scheme 7: A method of reducing uric acid levels and protecting kidney by using Lactobacillus paracasei LT12.

Technical scheme 8: A method of reducing uric acid levels and protecting kidney by using Lactobacillus plantarum CBT LP3.

Technical scheme 9: A method of reducing uric acid levels and protecting kidney by using a combination of Lactobacillus paracasei LT12 and Lactobacillus plantarum CBT LP3.

Technical scheme 10: A method according to any of technical schemes 1-9, wherein the composition is for use as a medicament or food.

Technical scheme 11: A composition comprising Lactobacillus paracasei LT12 and optionally Lactobacillus plantarum CBT LP3, in a weight ratio of 0.1-300:0.1-300, preferably 150:150.

Technical scheme 12: A composition according to technical scheme 11, wherein the composition has efficacy in reducing uric acid.

Technical scheme 13: A composition according to technical scheme 11, wherein the composition has efficacy in protecting kidneys.

Technical scheme 14: A composition according to technical scheme 11, wherein the composition has efficacy in immune modulation.

Technical scheme 15: A composition according to technical scheme 11, wherein the composition simultaneously has efficacies in reducing uric acid, protecting kidneys, and immune modulation.

Technical scheme 16: A medicament comprising a composition according to technical scheme 11.

Technical scheme 17: A medicament according to technical scheme 16, wherein the medicament comprises an effective amount of the composition.

Technical scheme 18: A medicament according to technical scheme 16 or 17, wherein the medicament comprises excipients, preferably the excipients comprise, but not limited to, one or more of sorbitol, maltodextrin, and magnesium stearate.

Technical scheme 19: A medicament according to any of technical schemes 16-18, wherein the medicament is a tablet, capsule, powder, or granule, but not limited thereto. Preparation of tablet, capsule, powder, or granule can be conducted according to general technique.

Technical scheme 20: A food comprising a composition according to technical scheme 11.

Under current technique, it is not found that Lactobacillus paracasei LT12 and Lactobacillus plantarum CBT LP3 have efficacies in reducing uric acid and protecting kidneys. In long term experiments, the inventors found that Lactobacillus paracasei LT12 and Lactobacillus plantarum CBT LP3 not only have efficacies in degrading uric acid precursor substances inosine and guanosin, but also reduce blood uric acid levels in mice having high uric acid levels. Specifically, a composition prepared from one or two of the strains, when fed to mice having high uric acid levels, can significantly reduce uric acid levels in having high uric acid levels. In addition, the efficacy in reducing uric acid is higher if both strains are used. Further, the composition has good protection on mice kidneys and can ameliorate renal damages caused by high uric acid levels.

In specific embodiments of the invention, Lactobacillus paracasei LT12 and Lactobacillus plantarum CBT LP3 can degrade uric acid precursor substances inosine and guanosin. That is, uric acid is not produced. Inosine and guanosine are themselves metabolized and utilized to produce other substances. Thus, uric acid production is reduced in the body. An efficacy in reducing uric acid is achieved.

It is known that high uric acid causes diseases or disorders comprising, but not limited to, hyperuricemia, gouty arthritis, kidney diseases, cardiovascular diseases, metabolic syndrome, stroke, neurodegenerative disorders, wherein the kidney diseases comprise acute uric acid nephropathy, chronic urate nephropathy, and uric acid nephrolithiasis; the cardiovascular diseases comprise vascular diseases, coronary heart disease, and hypertension; the metabolic syndrome comprises hypercholesterolemia; and the neurodegenerative disorders comprise Parkinson's disease, Huntington's disease, and multiple sclerosis. The present invention can achieve the efficacy in reducing uric acid, and thus can be useful in preventing, treating, improving or ameliorating one or more of the above diseases or disorders.

Lactobacillus paracasei LT12 (SHANGHAI LYTONE BIOCHEMICALS, LTD.) and Lactobacillus plantarum CBT LP3 (Cell Biotech Co., Ltd., South Korea) used in the invention are commercially available.

Lactobacillus paracasei LT12 has been deposited with the ARS Culture Collection (NRRL) on 21 September 2009, having an accession number of NRRL-B50327.

Lactobacillus plantarum CBT LP3 has been deposited with the Korean Collection for Type Cultures (KCTC), having an accession number of KCTC 10782BP.

Serum uric acid level is the most important indicator to directly determine hyperuricemia. Creatinine is formed from phosphocreatine through automatic and irreversible conversion in the muscle. Unless there is great change in muscle mass, the amount of creatinine formed under general condition is rather constant. Circulating amount of free creatinine depends completely on the rate of excretion. Thus, the amount of creatinine measured in the serum or plasma can be used for evaluating glomerular filtration rate to determine the status of renal function. Blood urea nitrogen originates from the liver and is passed through the kidney and excreted out of the body with urine. Renal failure, renal inflammation, and urinary tract obstruction can increase the amount of blood urea nitrogen.

In addition, when uric acid is produced from purine through catalysis by xanthine oxidase, large amounts of superoxide and H₂O₂ are generated, thereby causing systemic elevation of oxidative stress levels in the organism. Glutathione is an important endogenous anti-oxidant in the body, which can neutralize reactive oxygen species in the body. Superoxide dismutase is the most important antioxidant enzyme in the body. It can catalyze superoxide anion to undergo dismutation and generate hydrogen peroxide and oxygen. Glutathione peroxidase can use reduced glutathione to catalyze hydrogen peroxide and other organic oxides to produce water or organic alcohols. It can remove peroxides from living cells and plays a critical role in protecting cells from damage by free radicals. Lipids in the cells are susceptible to reaction with free radicals and produce lipid peroxides. Glutathione peroxidase can use reduced glutathione to reduce lipid peroxides, thereby eliminating the toxicity of free radicals. Glutathione peroxidase distributes in almost all the tissues. In some pathological conditions, the activities of glutathione peroxidase can be significantly upregulated or downregulated. Malondialdehyde is a natural product from oxidation of lipids in organisms. When animal or plant cells suffers from oxidative stress, lipids would be oxidized. The level of malondialdehyde reflects the level of oxidative stress in the body.

About ⅔ of the uric acids in an organism are excreted by the kidneys. The interaction between reabsorption and secretion of uric acid in the kidneys determines the amount of renal uric acid excretion. Various uric acid transporters participate in the transportation of uric acid in the kidneys. Among them, URAT1, GLUT9 and OAT1 are the most important transporter proteins URAT1 and GLUT9 are the most important transporter proteins for promoting uric acid reabsorption. URAT1 is an anion transporter protein expressed on the brush border membrane of renal proximal tubular cells, which can reabsorb extracellular uric acid into epithelial cells through secreting C1. GLUT9 is highly expressed in the basolateral membrane of renal proximal tubular cells, which is involved in the transportation of urate from the cytoplasm of renal tubular epithelial cells to the perivascular mesenchyme in a voltage dependent manner. Researches reveal that mutations of URAT1 and GLUT9 significantly increase uric acid excretion in humans. OAT1 located at the basolateral membrane of renal proximal tubular cells participates in uric acid excretion. Uric acid excretion is also mediated by ABCG2. Thus, OAT1 and ABCG2 are transporter proteins associated with uric acid excretion.

The technical schemes provided by the present invention produce the effect of reducing uric acid, protecting kidney, and immune modulation. Lactobacillus paracasei LT12 and Lactobacillus plantarum CBT LP3 can effectively degrade inosine and guanosine, resulting in decreased uric acid production. Mice experiments prove that mice with high uric acid levels, after continuous consumption of the composition of the present invention, have significantly lowered blood uric acid levels. In addition, compared to control group, renal damage is significantly reduced, indicating an improved efficacy in renal protection.

### BRIEF DESCRIPTION OF THE DRAWING

FIG. 1 shows the results of survival rate of probiotics in gastric acid tolerance tests.

FIG. 2 shows the animal experiment procedure.

FIG. 3 shows serum uric acid levels.

FIG. 4 shows serum creatinine levels.

FIG. 5 shows blood urea nitrogen levels.

FIG. 6 shows levels of the mice kidney inflammation factor TNF-α.

FIG. 7 shows levels of the mice kidney inflammation factor IL-1β.

FIG. 8 shows levels of the mice kidney inflammation factor IL-6.

FIG. 9 shows mice serum levels of glutathione.

FIG. 10 shows levels of glutathione in mice kidney tissues.

FIG. 11 shows mice serum levels of superoxide dismutase.

FIG. 12 shows levels of superoxide dismutase in mice kidney tissues.

FIG. 13 shows mice serum levels of glutathione peroxidase.

FIG. 14 shows levels of glutathione peroxidase in mice kidney tissues.

FIG. 15 shows mice serum levels of malondialdehyde.

FIG. 16 shows levels of malondialdehyde in mice kidney tissues.

FIG. 17 shows mice serum levels of xanthine oxidase.

FIG. 18 shows levels of xanthine oxidase in mice liver tissues.

FIG. 19 shows URAT1 expression levels in mice kidney tissues.

FIG. 20 shows GLUT9 expression levels in mice kidney tissues.

FIG. 21 shows OAT1 expression levels in mice kidney tissues.

FIG. 22 shows ABCG2 expression levels in mice kidney tissues.

FIG. 23 shows the inhibition rate of different Lactobacillus strains on xanthine oxidase.

### DETAILED DESCRIPTION OF THE INVENTION

For the purpose of a more clear understanding of the technical features, goals and advantages of the present invention, provided below are detailed descriptions of the technical schemes of the present invention. The example is offered for illustrative purposes only, and is not intended to limit the scope of the present invention in any way.

### EXAMPLE

### Example 1: Evaluations on the Ability of Probiotics to Degrade Inosine and Guanosine

Different probiotics (Lactobacillus plantarum CBT LP3, Lactobacillus paracasei LT12, Bifidobacterium bifidum (CBT BF3, Cell Biotech Co., Ltd., South Korea), Lactococcus lactis sp. (DUP-12752, BioSource Cultures and Flavors Inc., U.S.A.), Bifidobacterium longum (CBT BG7, Cell Biotech Co., Ltd., South Korea)) were activated. The cells were inoculated at an amount of 1% to MRS culture medium and passaged at 37° C. The cells were prepared for use after the second passage. Activated probiotics were diluted to 1×10⁹ CFU/mL using MRS, centrifuged at 4000 rpm for 10 minutes, and the supernatant was removed. The cells were washed with 0.9% NaCl 2-3 times and bacterial mire was obtained for subsequent experiments. Mires of different probiotics were separately added with 0.25 mg/mL inosine-guanosine reaction solution, incubated at 37° C. for 1 hour, and bathed in hot water for 5 minutes to terminate reactions. Samples were filtrated with 0.22 μm water phase membrane followed by detection with high performance liquid chromatography (HPLC). Inosine and guanosine degradation rates were calculated according to the following formula:

Degradation rate (%)=(A₀−A₁)/A₀×100%

In the formula, A₀ is the peak area of inosine or guanosine standards, A₁ is the peak area of inosine or guanosine after degradation.

High performance liquid chromatography (HPLC) specific detection method: mobile phase: A1: 20 mmol/L KH₂PO₄ (pH 3.0); B1: methanol; A1:B1=95:5. Column: Shimatsu 5020-03946 C18, detection wavelength: 254 nm, flow rate: 0.5 mL/mL.

The results obtained from Example 1 are shown in Table 1.

TABLE 1
inosine and guanosine degradation rate by probiotics

Degradation Rate

| Sample | Inosine | Guanosine |
|---|---|---|
| Lactobacillus paracasei LT12 | 35.60% | 36.21% |
| Lactobacillus plantarum CBT LP3 | 56.17% | 74.44% |
| Bifidobacterium bifidum CBT BF3 | 15.28% | 17.35% |
| Lactococcus lactis sp. DUP-12752 | 14.06% | 11.89% |
| Bifidobacterium longum CBT BG7 | 12.83% | 10.94% |

Upon comparison of the above results, it is proven that Lactobacillus paracasei LT12 and Lactobacillus plantarum CBT LP3 exhibit higher ability in degrading the precursors, i.e., inosine and guanosine, of uric acid, and thus, have potential in reducing uric acid. Therefore, Lactobacillus paracasei LT12 and Lactobacillus plantarum CBT LP3 were selected as the strains for testing uric acid reduction.

### Example 2: Identification of Metabolites from Probiotics

Probiotics can metabolize and produce short chain fatty acids when colonized in the intestines. Short chain fatty acids in turn promote growth and propagation of probiotics. In the intestines, the suitable growth pH for pathogenic bacteria or opportunistic pathogenic bacteria such as Escherichia coli, Staphylococcus, and Salmonella is 6.0-7.0. If being at a pH lower than 4.0, the bacterial is basically inactivated. The most suitable growth pH for probiotics is 3.0-4.5. Short chain fatty acids are present in the intestinal tract in the form of cations. When short chain fatty acids in the intestines increase, hydrogen ions can be released to lower the pH value in the intestines, thereby promoting the growth and propagation of intestinal probiotics and inhibiting the growth of pathogenic bacteria or opportunistic pathogenic bacteria such as Escherichia coli, Staphylococcus, and Salmonella. Thus, if the metabolites from probiotics contain short chain fatty acids, the probiotics can simultaneously promote the growth and propagation of other beneficial bacteria in the intestines and improve the balance of intestinal microbes.

Lactobacillus paracasei LT12 and Lactobacillus plantarum CBT LP3, which were selected for capable of degrading inosine and guanosine, were subjected to identification tests for metabolites. The probiotics were activated. The cells were inoculated at an amount of 1% to MRS culture medium and passaged at 37° C. The cells were taken out after the second passage, centrifuged at 3000 rpm for 10 minutes, and the supernatant was filtrated using 0.22 μm membrane to obtain metabolites from the probiotics. The results are shown in Table 2.

Standard curve plotting for short chain fatty acids: preparing certain amounts of acetic acid, propionic acid and butyric acid standard samples, diluting to 10 mM, 25 mM, 50 mM, 75 mM and 100 mM using distilled water, and inputting the samples to high performance liquid chromatography (HPLC) for detection and plotting the standard curve.

High performance liquid chromatography (HPLC) specific detection method: mobile phase: A1: 0.025% phosphoric acid; B1: acetonitrile; A1:B1=95:5. Column: Shimatsu 5020-03946 C18, detection wavelength: 210 nm, flow rate: 0.5 mL/mL.

TABLE 2
Identification of short chain fatty acids from probiotics.

| | acetic acid | propionic acid | butyric acid |
|---|---|---|---|
| Sample | (mmol/L) | (mmol/L) | (mmol/L) |
| Lactobacillus paracasei LT12 | 106.12 | 6.63 | 3.56 |
| 24 hours metabolites | | | |
| Lactobacillus paracasei LT12 | 107.10 | 6.35 | 3.84 |
| 48 hours metabolites | | | |
| Lactobacillus paracasei LT12 | 107.03 | 6.06 | 5.16 |
| 72 hours metabolites | | | |
| Lactobacillus plantarum CBT | 125.20 | 5.22 | 0.18 |
| LP3 | | | |
| 24 hours metabolites | | | |
| Lactobacillus plantarum CBT | 105.46 | 4.23 | 6.83 |
| LP3 | | | |
| 48 hours metabolites | | | |
| Lactobacillus plantarum CBT | 180.83 | 5.35 | 0.49 |
| LP3 | | | |
| 72 hours metabolites | | | |

The experiment proves that both Lactobacillus paracasei LT12 and Lactobacillus plantarum CBT LP3 can metabolize to produce short chain fatty acids, primarily acetic acid with some amounts of propionic acid and butyric acid. Thus, Lactobacillus paracasei LT12 and Lactobacillus plantarum CBT LP3 can promote the growth and propagation of beneficial bacteria in the intestines, and promote the propagation of beneficial bacteria that are naturally present in human intestines and exhibit the abilities to degrade inosine and guanosine. A synergistic effect in enhancing the reduction of uric acid can be achieved.

### Example 3: Gastric Acid Tolerance Test for Probiotics

Whether probiotics can safely colonize in human intestines or not is highly associated with their tolerance to gastric acid. Only the probiotics that safely pass the gastric acid have the chance to reach the intestines and colonize. Thus, tolerance to gastric acid is critical for determining whether probiotics can produce the desired efficacies in human bodies.

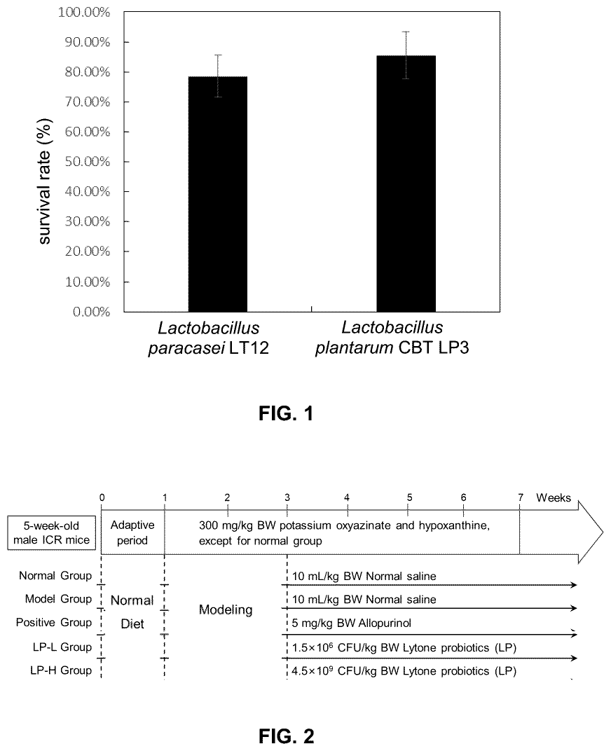

Lactobacillus paracasei LT12 and Lactobacillus plantarum CBT LP3, which were selected for capable of degrading inosine and guanosine, were subjected to tolerance test that simulates gastric fluid. The probiotics Lactobacillus paracasei LT12 and Lactobacillus plantarum CBT LP3 were activated. The cells were inoculated to MRS culture medium and passaged at 37° C. The cells were taken out after the second passage. Suitable amounts of bacterial solutions were collected and centrifuged at 5000 rpm for 10 minutes, and the supernatant was discarded to obtain the bacteria. The bacterial cells were re-suspended using sterile saline. According to the OD-CFU curve on hand, the OD values of each bacterial strain was adjusted so that the number of live bacteria in the suspension is maintained at around 1×10⁸CFU/mL, and collect the bacterial suspensions. Prepare 2 g NaCl and dissolve the same in 1L deionized water. Adjust the pH using 2M HCl to 2.5 and cool after autoclave. Prepare pepsin and add the same to the sterile solution for complete dissolution so that the final enzyme activity reaches 2000 U/mL. The solution was filtrated using 0.22 μm water phase membrane to obtain simulating gastric fluid. Certain volume of re-suspended bacterial solution was obtained and centrifuged at 5000 rpm for 10 minutes, and the supernatant was discarded. Equal volume of simulating gastric fluid was added and sufficiently re-suspended, and the solution is incubated at a temperature of 37° C., 80 rpm, for 2 h. After digestion by simulating gastric fluid, the solution was centrifuged at 5000 rpm for 10 minutes, and the supernatant was discarded. Equal volume of saline was added and sufficiently re-suspended. The number of live bacteria was counted using dilution spread method. The survival rate after digestion by simulating gastric fluid was calculated. The results are shown in FIG. 1.

The experiment proves that both Lactobacillus paracasei LT12 and Lactobacillus plantarum CBT LP3 are well tolerated to gastric acid. After digestion for 2 hours in simulating gastric fluid, the survival rate of Lactobacillus paracasei LT12 reaches 78%, and the survival rate of Lactobacillus plantarum CBT LP3 reaches 85%. The results prove that both Lactobacillus paracasei LT12 and Lactobacillus plantarum CBT LP3 have good ability in tolerating gastric acid, can safely reach the intestines to colonize in human bodies, and demonstrate the efficacies in reducing uric acid.

### Example 4: Test of Lactobacillus paracasei LT12 for Efficacies in Reducing Uric Acid and Protecting Kidneys

### 4.1 Animal Experimental Method

### 4.1.1 Experimental Animals Grouping and Treatments

Tested animals are 5-week old male ICR mice purchased from Shanghai JSJ Laboratory Animal Co., Ltd., SPF grade. All mice were grown under the conditions of a temperature of 24±2° C., alternating 12 h light and 12 h dark, and free feeding and drinking for adaptation. After 1 week of adaptation, mice were randomly grouped into 10 groups, 7 mice in one group. The groups are normal control group, model group, positive control group, low dose Lactobacillus paracasei LT12 group, and high dose Lactobacillus paracasei LT12 group (see Table 3). Specific gavage amounts and experimental design are as shown in FIG. 2. Modeling period is two weeks and interfering period is four weeks. During modeling, except for the normal control group, the other groups were subjected to gavage with 300 mg/kg potassium oxonate and hypoxanthine (formulated into a suspension using 0.5% CMC-Na solution) every day for high uric acid modeling. During interference, except for the normal control group, the other groups were subjected to gavage with 300 mg/kg potassium oxonate and hypoxanthine every day for continued modeling. 2 h after modeling, each group of mice were administered at the dose according to FIG. 2. The drug samples for each group were dissolved in saline. The same amount of saline was administered to normal groups and model group.

TABLE 3
Experimental groups

| | Abbreviation | Group name |
|---|---|---|
| Group 1 | Normal | Normal group |
| Group 2 | Model | Model group |
| Group 3 | Positive | Positive group |
| Group 4 | LP-L | Lactobacillus paracasei LT12 low dose group |
| Group 5 | LP-H | Lactobacillus paracasei LT12 high dose group |

### 4.1.2 Sampling and Treatments for Detection

During modeling, blood was collected through cutting the tail to detect changes in serum uric acid every week. At the last two days of the experiment, fresh feces of mice of each group were collected and kept in −20° C. refrigerator. Before sacrifice, mice were fasted for 12 h. The last modeling was conducted. Blood was collected 1 h after modeling followed by sacrifice and dissection. Mice whole blood is placed at 4° C. for 2 h followed by centrifugation at 3000 rpm for 10 min. The serum was aliquoted and stored in −80° C. refrigerator. Mice liver, kidneys, and colon were collected. One of the kidneys was preserved in 4% polyoxymethylene for pathological analysis. Other tissue samples were immediately freezed in liquid nitrogen and stored at −80 ° C. for preservation.

### 4.2 Detection Method

The levels of uric acid, creatinine, blood urea nitrogen, lactate dehydrogenase, xanthine oxidase (XOD), oxidative stress levels (glutathione (GSH), superoxide dismutase (SOD), glutathione peroxidase (GSH-Px), malondialdehyde (MDA), inflammatory factors (TNF-alpha, IL-1β, and IL-6)) were detected according to the detection method described in the specification in the kits. Pathological analysis on renal tissue were completed in corporation with Wuhan Servicebio Technology Co., Ltd., China. Immunoblotting analysis on renal uric acid transporter proteins were conducted according to internal methods in labs.

### 4.3 Data Processing Method

Data were subjected to statistical analysis using SPSS 22.0 software by Tukey method. p<0.05 is considered that the difference has statistical significance. GraphPad Prism 8 and R studio were used for plotting, analysis for relevance, and analysis for bioinformatics.

### 4.4 Experimental Results

### 4.4.1 Modeling Results

Generally, urate oxidase is present in mammals including mice. Being an inhibitor of urate oxidase, potassium oxonate is commonly used in modeling high uric acid animal models. Purine substances such as hypoxanthine are precursors of uric acid. Thus, hypoxanthine and potassium oxonate have broad applications in modeling secondary high uric acid mice model.

After two weeks of modeling, blood was collected through cutting the tail to detect mice serum uric acid levels. The results are shown in Table 4. After two weeks of modeling, except for the mice of the normal control group, serum uric acid levels of all other mice were significantly increased compared to mice of the normal group, with an increase of around two folds, which indicates that the high uric acid modeling was successful. Therefore, starting from week 3, modeling proceeds along with gavage for probiotics interference.

TABLE 4
Serum uric acid levels after modeling for two weeks

| | | 1 | 2 | 3 | 4 | 5 | 6 | 7 | Mean | SD |
|---|---|---|---|---|---|---|---|---|---|---|
| Group 1 | Normal | 95.2 | 104.1 | 126.4 | 104.1 | 95.2 | 113.0 | 86.2 | 103.5 | 13.2 |
| Group 2 | Model | 242.4 | 175.5 | 233.5 | 162.1 | 162.1 | 255.8 | 206.7 | 205.4 | 39.5 |
| Group 3 | Positive | 224.5 | 197.8 | 215.6 | 215.6 | 211.2 | 224.5 | 255.8 | 220.7 | 17.9 |
| Group 4 | LP-L | 233.5 | 153.2 | 233.5 | 184.4 | 242.4 | 179.9 | 171.0 | 199.7 | 35.9 |
| Group 5 | LP-H | 215.6 | 188.8 | 162.1 | 246.8 | 224.5 | 162.1 | 184.4 | 197.8 | 32.3 |

### 4.4.2 Effects of Different Interferences on Mice Renal Functions

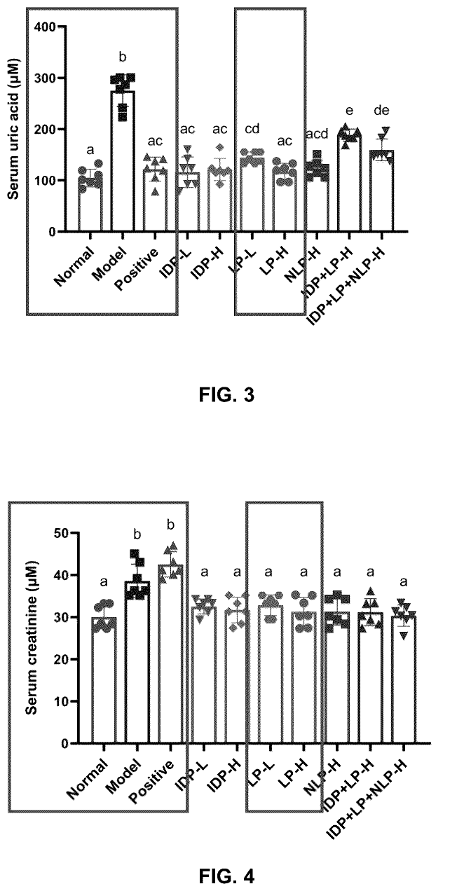

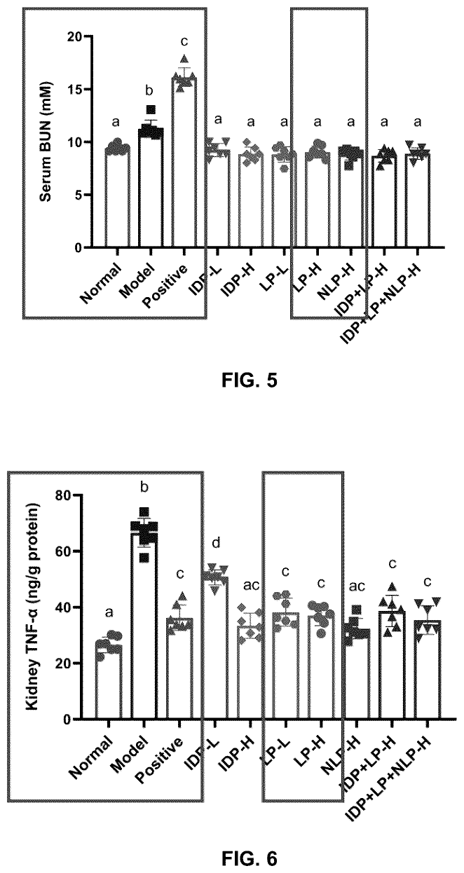

The detection results of three renal function indicators, i.e., uric acid, serum creatinine, and blood urea nitrogen, are shown in FIGS. 3-5.

With respect to uric acid levels, the uric acid levels of the modeling group are significantly higher than all other groups. After interference for four weeks, the uric acid levels of all the interfering groups were significantly reduced, which indicates that interferences by all the formulations yield satisfying effects in reducing uric acid. In addition, the uric acid levels of the positive control group, and Lactobacillus paracasei LT12 low and high dose groups reduced to the levels of the normal group (no significant difference from normal group). Although the uric acid levels of the Lactobacillus paracasei LT12 low dose group were slightly higher than the normal group, said level is significantly lower than the model group.

As the uric acid concentrations of the model group increase, the levels of serum creatinine and blood urea nitrogen significantly increase as well, compared to mice of the normal group. The results are consistent with those reported in references. That is, hyperuricemia accompanies with damage in renal function. Except for the positive control group, serum creatinine and blood urea nitrogen levels of all other interfering groups significantly reduced to normal levels (no significant difference from the normal group, with significant difference from the model group), indicating that Lactobacillus paracasei LT12 can ameliorate renal damage caused by hyperuricemia. Creatinine and blood urea nitrogen levels of the mice of the positive control group increased, which indicates that although allopurinol can reduce the uric acid levels of high uric acid mice, strong renal toxicity was generated to the mice.

### 4.4.3 Protection of Kidneys in Mice by Different Interferences

By modeling for high uric acid using potassium oxonate and hypoxanthine, apparent inflammation and damages in tissue structure were present in mice kidneys. The efficacies of resorption by renal tubules and filtration by renal glomerulus have great impact on uric acid levels. Damages in renal tubules and disorder in the filtration function of renal glomerulus would further increase uric acid concentrations, leading to deposits of uric acid crystals. Although allopurinol treatment reduced uric acid levels of high uric acid mice, severe renal damages were caused. The Lactobacillus paracasei LT12 groups significantly improved damages in renal tissues caused by hyperuricemia and ameliorated inflammatory cell infiltration in renal tissues.

TABLE 5
Pathological scores of mice kidneys

| | | | renal | | | | | |
|---|---|---|---|---|---|---|---|---|
| | | inflammatory | tubules | renal | renal | connective | | |
| | | cell | hydropic | tubules | tubules | tissue | Protein | Total |
| Number | | infiltration | degeneration | atrophy | dilation | proliferation | cast | score |
| Normal | 1-1 | 0 | 0 | 0 | 0 | 0 | 0 | 0 |
| | 1-2 | 0 | 0 | 0 | 0 | 0 | 0 | |
| | 1-3 | 0 | 0 | 0 | 0 | 0 | 0 | |
| | 1-4 | 0 | 0 | 0 | 0 | 0 | 0 | |
| | 1-5 | 0 | 0 | 0 | 0 | 0 | 0 | |
| | 1-6 | 0 | 0 | 0 | 0 | 0 | 0 | |
| | 1-7 | 0 | 0 | 0 | 0 | 0 | 0 | |
| Model | 2-1 | 1 | 1 | 0 | 0 | 0 | 0 | 16 |
| | 2-2 | 1 | 1 | 0 | 1 | 0 | 0 | |
| | 2-3 | 1 | 1 | 0 | 0 | 0 | 0 | |
| | 2-4 | 1 | 1 | 0 | 0 | 0 | 0 | |
| | 2-5 | 0 | 2 | 0 | 0 | 0 | 0 | |
| | 2-6 | 1 | 2 | 0 | 0 | 0 | 0 | |
| | 2-7 | 1 | 1 | 0 | 0 | 0 | 0 | |
| Positive | 3-1 | 0 | 1 | 0 | 0 | 0 | 1 | 22 |
| | 3-2 | 0 | 1 | 0 | 1 | 0 | 0 | |
| | 3-3 | 1 | 1 | 0 | 0 | 0 | 0 | |
| | 3-4 | 1 | 1 | 0 | 0 | 0 | 0 | |
| | 3-5 | 1 | 0 | 0 | 1 | 0 | 0 | |
| | 3-6 | 1 | 2 | 1 | 1 | 1 | 0 | |
| | 3-7 | 1 | 2 | 1 | 1 | 1 | 0 | |
| LP-L | 6-1 | 0 | 1 | 0 | 0 | 0 | 0 | 4 |
| | 6-2 | 0 | 0 | 0 | 0 | 0 | 0 | |
| | 6-3 | 0 | 1 | 0 | 0 | 0 | 0 | |
| | 6-4 | 0 | 1 | 0 | 0 | 0 | 0 | |
| | 6-5 | 0 | 0 | 0 | 0 | 0 | 0 | |
| | 6-6 | 0 | 1 | 0 | 0 | 0 | 0 | |
| | 6-7 | 0 | 0 | 0 | 0 | 0 | 0 | |
| LP-H | 7-1 | 0 | 0 | 0 | 0 | 0 | 0 | 2 |
| | 7-2 | 0 | 1 | 0 | 0 | 0 | 0 | |
| | 7-3 | 0 | 1 | 0 | 0 | 0 | 0 | |
| | 7-4 | 0 | 0 | 0 | 0 | 0 | 0 | |
| | 7-5 | 0 | 0 | 0 | 0 | 0 | 0 | |
| | 7-6 | 0 | 0 | 0 | 0 | 0 | 0 | |
| | 7-7 | 0 | 0 | 0 | 0 | 0 | 0 | |

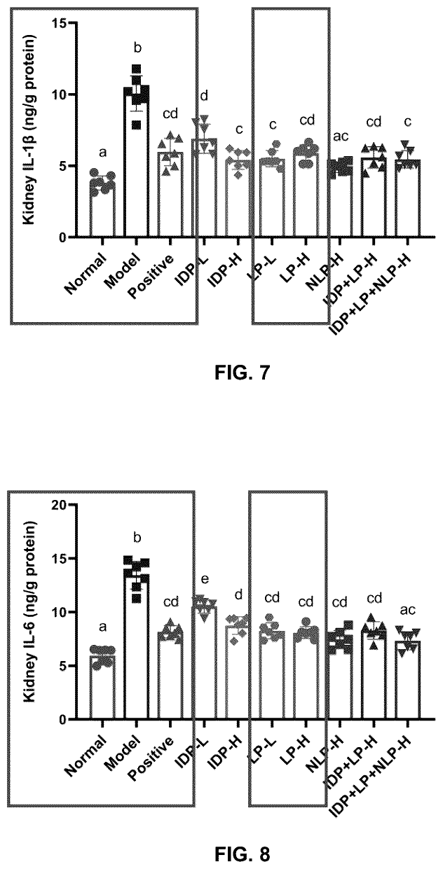

After pathological analysis on kidneys, further studies were conducted for the effect of hyperuricemia induced by hypoxanthine and potassium oxonate on various cytokine levels in mice renal tissues. As shown in FIGS. 6-8, after modeling for six weeks, the levels of pro-inflammatory factors (TNF-α, IL-1β, and IL-6) in the kidneys of the mice of the model group significantly increased compared to the normal group, which indicates that modeling causes significant inflammatory responses in mice kidneys. After various interferences, the levels of the three pro-inflammatory factors in all the interfering groups were significantly reduced.

### 4.4.4 Improvement of Oxidative Stress in Mice by Different Interferences

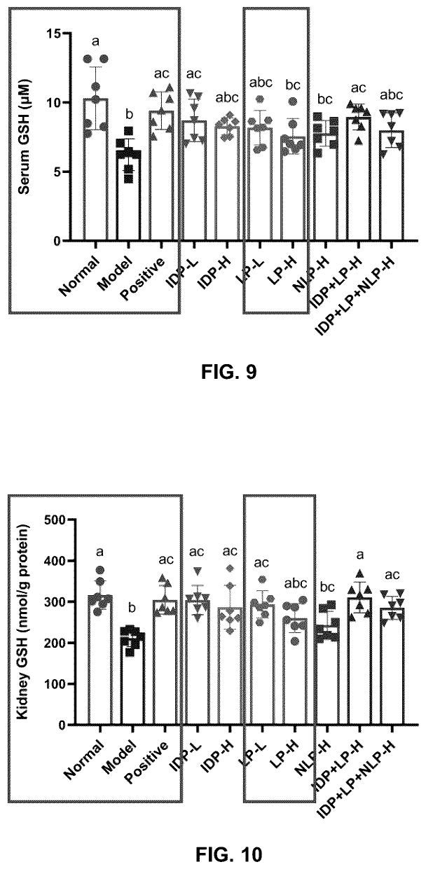

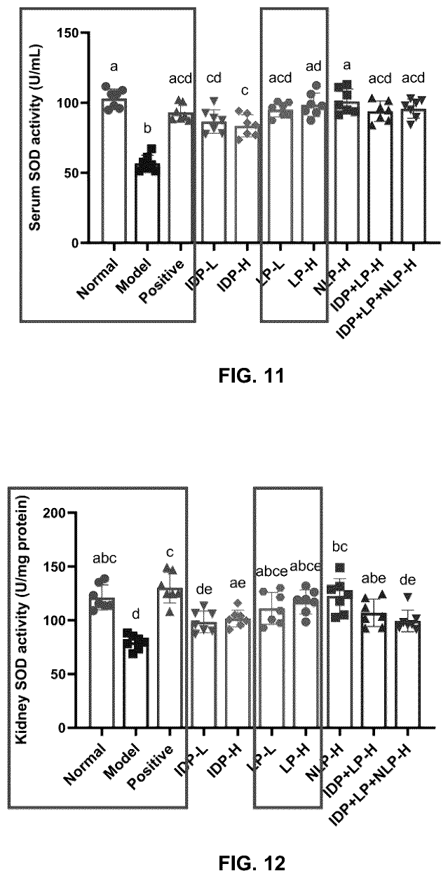

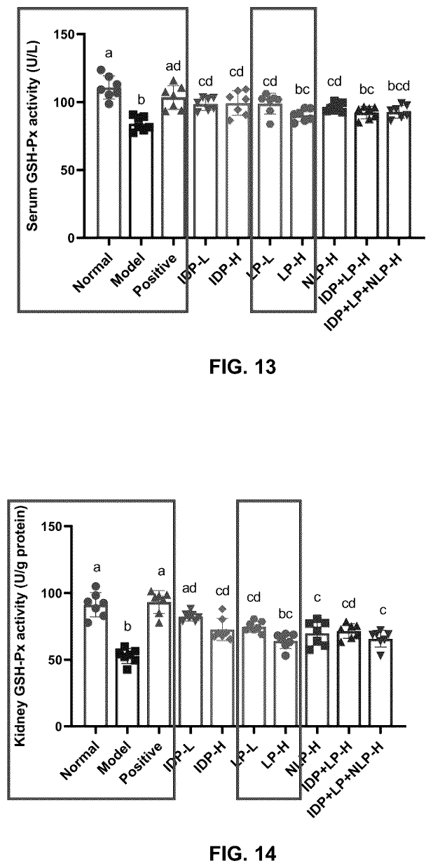

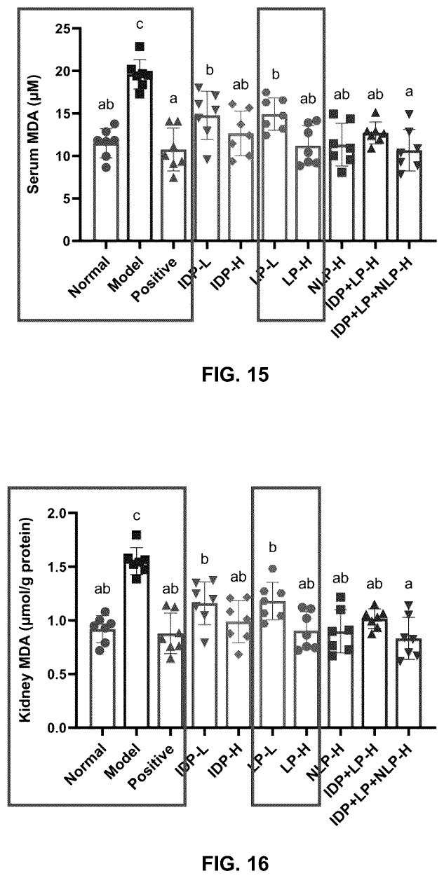

The results of detection of glutathione (FIGS. 9 and 10), superoxide dismutase (FIGS. 11 and 12), glutathione peroxidase (FIGS. 13 and 14), and malondialdehyde (FIGS. 15 and 16) in serum and renal tissue lysate show that levels of glutathione, superoxide dismutase, and glutathione peroxidase in the model group were significantly lower than the normal group, and the level of malondialdehyde is significantly higher than the normal group. All treatments increase the levels of glutathione, superoxide dismutase, and glutathione peroxidase in the serum and kidney tissues of the mice of the high uric acid model, and significantly reduce the level of malondialdehyde in the serum and kidney tissues of the mice of the high uric acid model. The results indicate that all treatments can ameliorate oxidative damages caused by uric acid production.

From the above detection results, it is found that Lactobacillus paracasei LT12 can ameliorate oxidative damages caused by uric acid production.

4.4.5 Effect of Different Interferences on Critical Enzymes Associated with Uric Acid Synthesis in Mice

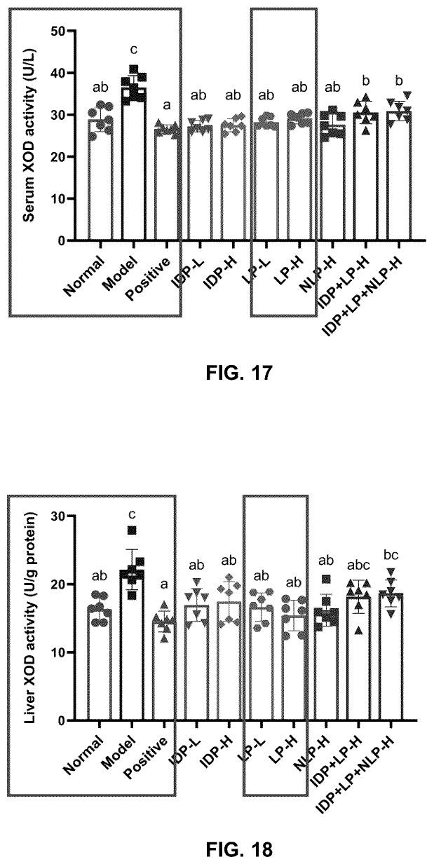

Xanthine is the critical enzyme in uric acid production. Xanthine oxidase is primarily expressed in the liver. The activities of xanthine oxidase in serum and liver of mice of each group are shown in FIGS. 17 and 18. Gavage with hypoxanthine caused accumulation of great amount of uric acid precursor substances in mice body, thereby causing the activities of xanthine oxidase in serum and liver of mice of the model group to be significantly higher than normal mice. Allopurinol is an inhibitor of xanthine oxidase. Thus, the activities of xanthine oxidase in serum and liver of mice of the positive control group are the lowest, which is lower than the activities of xanthine oxidase in liver of the normal group, though there is no significant difference. From the results, it is found that the efficacy of Lactobacillus paracasei LT12 is similar to allopurinol, and can effectively inhibit xanthine oxidase activities and reduce uric acid levels.

### 4.4.6 Effect of Different Interferences on Mice Uric Acid Transporter Proteins

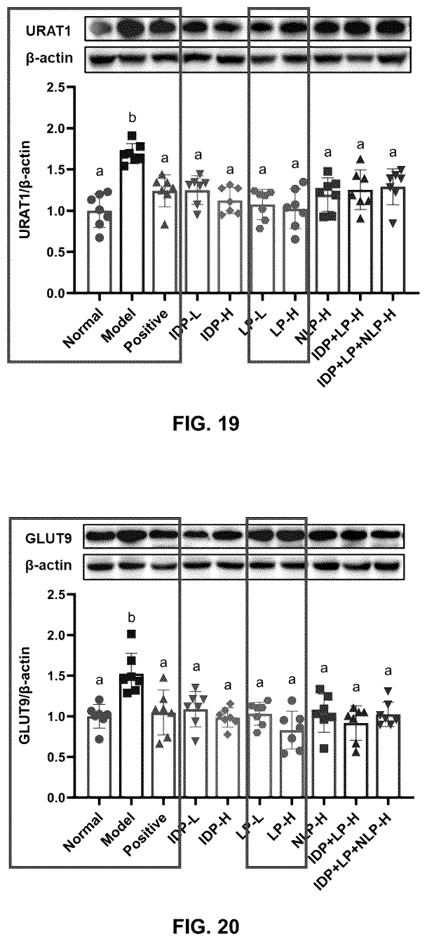

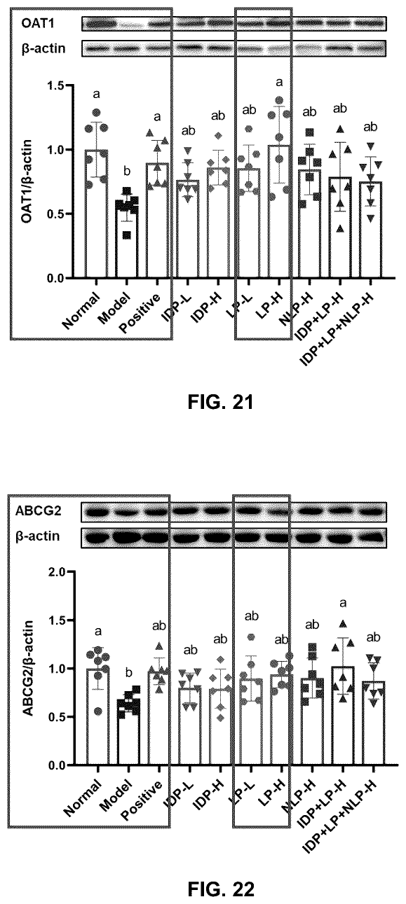

The detection results of the expression levels of the transporter proteins in renal tissue lysates are shown in FIGS. 19-22. The expression levels of URAT1 and GLUT9 in mice of the model group were significantly higher than the normal group. All treatments significantly decreased the expression levels of URAT1 and GLUT9 in renal tissues of mice of the high uric acid model, thereby decreasing uric acid resorption. The expression levels of OAT1 and ABCG2 in mice of the model group were significantly lower than the normal group. All treatments increased the expression levels of OAT1 and ABCG2 in renal tissues of mice of the high uric acid model, thereby promoting uric acid excretion.

### Example 5: Comparison of Lactobacillus paracasei LT12 on Efficacy in Reducing Uric Acid with Other Lactobacillus strains

Comparison on the efficacy in inhibiting xanthine oxidase (XO) by Lactobacillus paracasei LT12, Lactobacillus acidophilus DDS-1, (ATCC Number SD5212), and Lactobacillus rhamnosus (ATCC Number SD5217) was conducted. Lactobacillus rhamnosus, Lactobacillus acidophilus DDS-1 and Lactobacillus paracasei LT12 were cultured in MRS agar at 36° C. under anaerobic condition for 3 days. After passages twice, the cells were inoculated with single colony to MRS broth and cultured for 1 day followed by transferring to a culture medium and culture at room temperature for 2-3 days (the number of Lactobacillus bacteria is around 1.8×10⁹). The culture medium was centrifuged at 5000 rpm for 10 minutes. Culture medium of each strain of Lactobacillus bacteria were isolated as samples for subsequent experiment.

1 mL sample was homogeneously mixed with 3 mL reaction solution (0.1 mL, 0.2 units/mL XO) and 2.9 mL 50 mM phosphate buffer (pH 7.5), reacted in 25° C. water bath for 20 min, followed by adding 2 mL substance solution (150 mM, xanthine (X)). The solution was kept in 25° C. water bath for 30 min. Reaction was terminated by adding 1 mL 1N HCl.

The above reaction solution was filtrated with 0.2 μm filtration membrane and subjected to HPLC analysis. The analytical conditions were as follows:
1. Column: Thermo ODS Hypersil Part No. 30105-254630 and SUPELCO 516 C-18 cat #50302-U 25 cm×4.6 mm
2. Mobile phase: 50 mM KH₂PO₄, pH 7.5
3. Flow rate: 1 mL/min
4. Column temperature: 37° C.
5. Injection volume: 20 μL (+wash)
6. Detector: UV 290 nm
7. Analysis time: 10 min

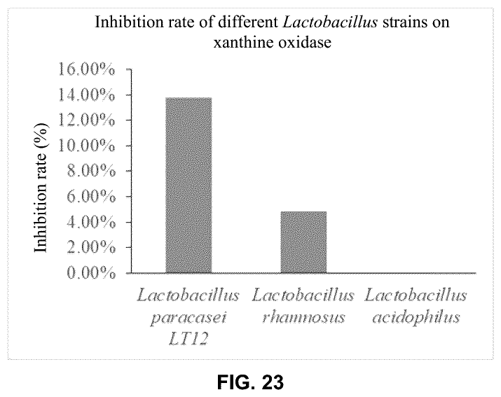

The results are shown in FIG. 23 and the table below:

| Lactobacillus bacteria | Inhibition rate (%) |
|---|---|
| Lactobacillus paracasei LT12 | 13.79% |
| Lactobacillus rhamnosus | 4.85% |
| Lactobacillus acidophilus DDS-1 | 0.00% |

From the results of analysis in reducing uric acid, it is found that Lactobacillus paracasei LT12 has the best inhibition rate and the next is Lactobacillus rhamnosus. Lactobacillus acidophilus DDS-1 has almost no detectable inhibition. From the above, results, it is known that even for Lactobacillus bacteria under the same Genus, the activities of different strains are still different from each other. It was unexpectedly found in the present invention that Lactobacillus paracasei LT12 has superior XO inhibitory activities compared to other Lactobacillus bacteria strains. Thus, the functions of Lactobacillus paracasei LT12 cannot be deduced from those of other Lactobacillus bacteria strains.

### Embodiment 1

Provided is a composition comprising, in weight ratio:

| | |
|---|---|
| Lactobacillus paracasei LT12 | 0.1-300 part |
| Lactobacillus plantarum CBT LP3 | 0.1-300 part |

### Embodiment 2

Provided is a composition comprising, in weight ratio:

| | |
|---|---|
| Lactobacillus paracasei LT12 | 150 part |
| Lactobacillus plantarum CBT LP3 | 150 part |

### Embodiment 3

Provided is a medicament comprising a composition of Embodiment 1 or 2

Provided is a medicament having efficacy in reducing uric acid, protecting kidneys, and immune modulation.

Provided is a medicament for use in preventing, treating, improving, or ameliorating diseases or disorders caused by high uric acid levels, including but not limited to, hyperuricemia, gouty arthritis, kidney diseases, cardiovascular diseases, metabolic syndrome, stroke, neurodegenerative disorders, wherein the kidney diseases comprise acute uric acid nephropathy, chronic urate nephropathy, and uric acid nephrolithiasis; the cardiovascular diseases comprise vascular diseases, coronary heart disease, and hypertension; the metabolic syndrome comprises hypercholesterolemia; and the neurodegenerative disorders comprise Parkinson's disease, Huntington's disease, and multiple sclerosis.

### Embodiment 4

Provided is a food comprising a composition of Embodiment 1 or 2

Provided is a food which serves as food supplement.

Provided is a food having efficacy in reducing uric acid, protecting kidneys, and immune modulation.

Provided is a food for use in improving, or ameliorating diseases or disorders caused by high uric acid levels, including but not limited to, hyperuricemia, gouty arthritis, kidney diseases, cardiovascular diseases, metabolic syndrome, stroke, neurodegenerative disorders, wherein the kidney diseases comprise acute uric acid nephropathy, chronic urate nephropathy, and uric acid nephrolithiasis; the cardiovascular diseases comprise vascular diseases, coronary heart disease, and hypertension; the metabolic syndrome comprises hypercholesterolemia; and the neurodegenerative disorders comprise Parkinson's disease, Huntington's disease, and multiple sclerosis.

What is claimed is:

1. A method for reducing uric acid, protecting kidneys and immune modulation, comprising administering a composition comprising an effective amount of Lactobacillus paracasei LT12 to a subject in need thereof.

2. The method of claim 1, wherein the Lactobacillus paracasei LT12 reduces levels of serum creatinine, blood urea nitrogen, or both.

3. The method of claim 1, wherein the Lactobacillus paracasei LT12 ameliorates renal damages caused by hyperuricemia.

4. The method of claim 1, wherein the Lactobacillus paracasei LT12 ameliorates inflammatory cell infiltration in renal tissues.

5. The method of claim 1, wherein the Lactobacillus paracasei LT12 reduces renal pro-inflammatory factor levels.

6. The method of claim 5, wherein the renal pro-inflammatory factor comprises TNF-α, IL-1β and IL-6.

7. The method of claim 1, wherein the Lactobacillus paracasei LT12 reduces oxidative stress.

8. The method of claim 1, wherein the Lactobacillus paracasei LT12 increases glutathione, superoxide dismutase, or glutathione peroxidase levels.

9. The method of claim 1, wherein the Lactobacillus paracasei LT12 ameliorates oxidative damages caused by uric acid.

10. The method of claim 1, wherein the Lactobacillus paracasei LT12 inhibits uric acid synthesis.

11. The method of claim 10, wherein the Lactobacillus paracasei LT12 inhibits xanthine oxidase activities.

12. The method of claim 1, wherein the Lactobacillus paracasei LT12 reduces uric acid resorption or promotes uric acid excretion.

13. The method of claim 12, wherein the Lactobacillus paracasei LT12 reduces expression levels of URAT1 and GLUT9 in renal tissues.

14. The method of claim 12, wherein the Lactobacillus paracasei LT12 increases expression levels of OAT1 and ABCG2 in renal tissues.

15. The method of claim 1, wherein the composition further comprises Lactobacillus plantarum CBT LP3.

16. A method for reducing uric acid, protecting kidneys and/or immune modulation comprising administering a composition comprising an effective amount of Lactobacillus plantarum CBT LP3 to a subject in need thereof.

17. The method of claim 16, wherein the composition further comprises Lactobacillus paracasei LT12.

18. The method of claim 1, wherein the composition is prepared as a drug or food.

19. The method of claim 18, wherein the drug is a tablet, capsule, powder, or granule.

20. The method of claim 18, wherein the food is a food supplement.

21. The method of claim 1, wherein administering the composition prevents, treats, improves, or ameliorates diseases or disorders caused by high uric acid level.

22. The method of claim 21, wherein the diseases or disorders caused by high uric acid level comprise hyperuricemia, gouty arthritis, kidney diseases, cardiovascular diseases, metabolic syndrome, stroke, neurodegenerative disorders.

23. The method of claim 22, wherein the kidney diseases comprise acute uric acid nephropathy, chronic urate nephropathy, and uric acid nephrolithiasis; the cardiovascular diseases comprise vascular diseases, coronary heart disease, and hypertension; the metabolic syndrome comprises hypercholesterolemia; and the neurodegenerative disorders comprise Parkinson's disease, Huntington's disease, and multiple sclerosis.
